(19) United States
(12) Patent Application Publication
(10) Pub. No.: US 2005/0027120 A1
(43) Pub. Date: Feb. 3, 2005

(54) Method for the synthesis of amides and related products from esters or ester-like compounds

(75) Inventor: Gabriel Gojon-Zorrilla, San Pedro Garza-Garcia (MX)

Correspondence Address: BACON & THOMAS, PLLC, 625 SLATERS LANE, FOURTH FLOOR, ALEXANDRIA, VA, 22314, US

(73) Assignee: REACTIMEX, S.A. DE C.V., Monterrey (MX)

(21) Appl. No.: 10/858,057

(22) Filed: Jun. 2, 2004

Related U.S. Application Data
(60) Provisional application No. 60/474,785, filed on Jun. 2, 2003.

(51) Int. Cl. C07D 265/36; C07D 215/16; C07D 263/52; C07D 239/02

(52) U.S. Cl. 544/105; 564/134; 544/224; 546/153; 548/217; 544/239; 544/309; 546/290

(57) ABSTRACT

A versatile, eco-friendly, and efficient method for the convenient conversion of esters and ester-like compounds into amides, peptides, carbamates, ureas, oxamides, oxamates, hydrazides, oxazolidinones, pyrazolones, oxazolidinediones, barbituric acids, and other molecules containing one or more OCN moieties in the presence of a diol or polyol is disclosed.

### CROSS-REFERENCE TO RELATED APPLICATION

This application claims the benefit of U.S. Provisional Application Ser. No. 60/474,785 filed Jun. 2, 2003 under 35 USC 119(e). The entire disclosure of this provisional application is herein incorporated by reference.

### BACKGROUND OF THE INVENTION

1. Field of the Invention

The present invention relates to the field of synthetic organic chemistry, and in particular to the synthesis of compounds whose molecules contain one or more OCN moieties such as amides, peptides, oxamates, oxamides, hydrazides, carbamates, ureas, oxazolidinones, pyrazolones and barbituric acids.

2. Description of Related Art

Carboxamides, peptides, carbamates, substituted ureas, hydrazides, barbituric acids, and other families of organic compounds whose molecules contain one or more OCN moieties comprise very many commercially important drugs, agrochemicals (insecticides, herbicides, etc.) and nutraceuticals.

The ammonolysis/aminolysis of carboxylic acid chlorides and anhydrides is the most frequently used-and most generally applicable-method for the preparation of carboxamides, both in the laboratory and in industry.

Ammonolysis of an acid chloride or anhydride yields unsubstituted amides (primary amides) R—CO—NH2. In a similar vein, aminolysis an acid chloride or anhydride using a primary amine gives N-substituted amides (secondary amides) R—CO—NHR′, and when a secondary amine is used the aminolysis yields N,N-disubstituted amides (tertiary amides), R—CO—NR′12

However, most acid chlorides and anhydrides are toxic and/or corrosive; their synthesis usually involves the use of even more toxic/corrosive inorganic compounds (thionyl chloride, phosphorus chlorides) which in turn are derived from elemental chlorine. All of these compounds are dangerous and must be handled and stored with extreme care on account of their reactivity towards water and of the irritant/corrosive nature of their hydrolysis products.

Furthermore, the reaction of acyl halides with ammonia or amine liberates hydrogen chloride, a highly corrosive and noxious chemical which is usually disposed of by neutralization with aqueous alkali thereby producing aqueous effluents whose treatment adds to process costs.

If the widely used Schotten-Baumann procedure is applied, aqueous effluents containing elevated salt loadings are generated too.

When acetyl chloride or acetic anhydride (the two most important acylating agents) are employed, the reaction is highly exothermic and must be carefully controlled, usually by cooling or dilution [Smith, M. B.; March, J. “March's Advanced Organic Chemistry”, fifth edition, Wiley-interscience, New York, 2001, p506]. The danger of a sudden temperature increase, especially when large scale reactions are run, must always be guarded against.

Finally, by-product formation (imides, ketene dimers) can complicate separations and reduce yields when anhydrides are used to acylate ammonia or primary amines and whenever acyl halides are employed.

On the other hand, acylations of ammonia or amines by carboxylic acid esters (i.e. ester ammonolysis or aminolysis) at atmospheric pressure is a method of amide synthesis of rather limited scope at the present time.

Thus, “the aminolysis of inactivated esters is known to be a difficult reaction, though it potentially constitutes a useful synthetic method as shown by the number of ways devised to facilitate it; uncatalysed aminolysis by primary amines requires temperatures higher than 200° C., whereas the corresponding reaction with secondary amines has never been reported [Matsumoto, K. et al “Direct aminolysis of inactivated and thermally unstable esters at high pressure” Chem. Ber. 122, 1357-1363 (1989)], “ester aminolysis, in general, occurs under harsh conditions that require high temperatures and extended reaction periods” [Varma, R. S.; Naicker, K. P. “Solvent-free synthesis of amides from non-enolizable esters and amines using microwave irradiation” Tetrahedron Letters, 40, 6177-6180 (1999)], and “The aminolysis of esters is generally a sluggish reaction unless esters having good leaving groups are used” [Hoegberg, T. et al, “Cyanide as an efficient and mild catalyst in the aminolysis of esters” J. Org. Chem. 52, 2033-2036 (1987)].

Accordingly, ester ammonolysis/aminolysis is very seldom used, especially in large-scale processes, although it has many advantages:
1) Most carboxylic acids can be easily converted into methyl or ethyl esters.
2) The reactions between an ester and ammonia or an amine are not highly exothermic; therefore, they are safer to run-especially in industry.
3) Esters are generally much less toxic and much safer to handle/store than acid halides and anhydrides
4) The acylation of ammonia or an amine by methyl or ethyl esters yields an amide and an alcohol as the only reaction products.
5) The by-product alcohol can be recycled (into ester), therefore the process is environmentally friendly
6) It is possible to ammonolyze/aminolyze enolyzable esters, hydroxyesters, and mercapto-substituted esters
7) Monoacylation of an aliphatic diamine can be carried out with higher selectivity when an ester is used as acylating agent
8) The ammonolysis/aminolysis of an ester is the first step in important reaction sequences that eventually lead to heterocycles such as oxazolinones, oxazolidinones, oxazolidinediones, benzisoxazoles, benzimidazoles, pyrazolones, pyrazolidindiones, dihydrooxazinediones, barbituric acids, thiobarbituric acids, benzoxazoles, benzothiazoles, quinolones, pyridazinones, pyridones, hydroxypyrimidines, dihydroxypyrimidines and thiazoles.

It is therefore clear that if the scope of ester ammonolysis/aminolysis were expanded, many synthetic amide producers would stop using acyl halides or acid anhydrides as acylating agents and turn to esters instead. It is also clear that synthetic sequences devised for obtaining new amides would undoubtedly favor ester ammonolysis/aminolysis over other acylation methods on account of its superior convenience, efficiency, environmental friendliness, and safety.

The present invention addresses this desideratum by providing an improved method for ester ammonolysis/aminolysis and related reactions that greatly expands their scope and usefulness.

### OBJECTS OF THE INVENTION

To provide an improved method applicable to the synthesis of amides through ammonolysis/aminolysis of esters, lactones, gem-diacyloxy derivatives, and other ester-like compounds.

To provide an improved method applicable to the synthesis of heterocyclic compounds that contain the OCN moiety, such as lactams, oxazolidinones, pyrazolones, oxazolidinediones, and barbituric acids from esters or ester-like compounds through cyclocondensation reactions involving said esters/ester-like compounds, ammonia or amines and, optionally a co-catalyst such as an alkaline metal carbonate or alkoxide.

To provide an improved method applicable to the synthesis of carbamates, ureas, oxamates, oxamides, and hydrazides from esters or ester-like compounds.

To provide an improved method aimed at liberating alcohols from their esters through ammonolysis/aminolysis.

To provide an improved method applicable to the synthesis of compounds containing one or more OCN moieties from esters/ester-like compounds and ammonia (or an ammonia precursor) or an amine/amine precursor or any amine like compound.

To provide an improved transamidation method.

To provide an improved method applicable to the synthesis of compounds whose molecule contain two or more OCN moieties by ammonolysis/aminolysis of esters derived from dicarboxylic or polycarboxylic acids.

To provide an improved method applicable to the synthesis of compounds whose molecules contain two or more OCN moieties through reaction of esters or ester-like compounds with diamines or polyamines.

### SUMMARY OF THE INVENTION

This applicant has unexpectedly discovered that some diols (especially 1,2-diols) and polyols catalyze the ammonolysis, aminolysis, and hydrazinolysis of esters and ester-like compounds via a catalytic cycle involving a transesterification reaction between the ester (or ester-like compound) and the diol/polyol. This discovery significantly widens the scope and heightens the usefulness of amide synthesis via ester ammonolysis/aminolysis; it also enhances the usefulness of a number of methods in synthetic heterocyclic chemistry which aim at the construction of rings containing the OCN moiety through cyclocondensation reactions. Furthermore, it is shown that superior synthetic methods based on the use of diols/polyols as catalysts/cocatalysts and/or solvents are applicable not only to the preparation of amides but also of carbamates, ureas, oxamides, oxamates, hydrazides and other similar molecules. Finally, it has been established that the same diols/polyols that catalyze the above-mentioned reactions can be advantageously used to catalyze or co-catalyze related chemical transformations such as transamidations.

Definitions

By “Ester-like compound” is meant any organic compound whose molecules contain one or more CO₂C moieties such as lactones-, gem-diacyloxy derivatives, and acetonides derived from alpha-hydroxyacids; said molecules may optionally comprise other functional groups.

By “Amine” is meant any substance whose molecules contain a CNH2 or CNHC moiety, regardless of the presence or absence of other functional groups.

By “Ammonia precursor” is meant any substance capable of generating ammonia “in situ”, such as urea-, ammonium carbonate, ammonium carbamate, etc. upon heating.

By “Amine precursor” is meant any substance capable of generating a primary or secondary amine “in situ”, such as “DIMCARB” (N,N-dimethylammonium N,N-dimethylcarbamate) when heated.

By “Hydrazine precursor” is meant any substance capable of generating hydrazine “in situ”, such as hydrazine monohydrate.

By “Substituted hydrazine” is meant any hydrazine derivative wherein 1, 2 or 3 hydrogen atoms of the hydrazine molecule have been replaced by alkyl radicals and/or aryl radicals and/or heteroaryl radicals, regardless of the presence or absence of functional groups on said radicals.

By “Substituted hydrazine precursor” is meant any substance capable of generating a “substituted hydrazine” “in situ”

By “Diol” is meant any substance whose molecules contain two alcoholic hydroxyl (OH) functional groups, regardless of the presence or absence of other functional groups.

By “Polyol” is meant any substance whose molecules contain 3 or more alcoholic hydroxyl (OH) functional groups, regardless of the presence or absence of other functional groups.

By “Diamine” is meant any substance whose molecules contain (attached to carbon atoms) two NH2 moieties or two NH moieties or one of each kind of moiety regardless of the presence or absence of other functional groups.

By “Polyamine” is meant any substance whose molecules contain (attached to carbon atoms) 3 or more NH2 or NH moieties in any possible combination, regardless of the presence or absence of other functional groups.

By “Amine-like compound” is meant any substance whose molecules contain an NH2 or NH moiety, which possesses chemical properties similar to those of a primary or secondary amine: hydrazine would be an example.

### DETAILED DESCRIPTION OF THE INVENTION

The instant method usually involves reacting one or more esters or ester-like compounds with one ore more amines or amine-like compounds in the presence of one or more diols/polyols, optionally in the presence of a co-catalyst (a metal, metal alkoxide, metal carbonate, metal cyanide, enzyme, tertiary amine, or any transesterification catalyst).

Many variations on this basic process are possible and are, of course, within the scope of the present invention. For instance:

1) Two stages might be employed: an initial transesterification step involving only the ester or ester-like compound, the diol/polyol and (optionally) a transesterification catalyst, with or without separation of by-product alcohol, and a final step wherein the initially obtained hydroxyester reacts with the amine or amine-like compound.

2) The process might involve recycling of the mother liquor obtained after separating the amide.

3) The process might involve superatmospheric or sub atmospheric pressures, high or low temperatures, use of inert solvents, inert atmospheres, etc.

4) Instead of using an ester and an amine, an aminoester might be used as starting material.

The effectiveness of a series of diols/polyols as nucleophilic catalysts in the acylation of monoethanolamine by ethyl acetate (using standardized conditions) was found to be:

Ethylene glycol>2,2-dimethyl-1,3-propanediol>glycerol>propylene glycol>D-sorbitol>blank (no catalyst present)˜diethylene glycol>1,3-propanediol˜1,4-butanediol.

Therefore, ethyleneglycol is the preferred catalyst/solvent, but the use of 2,2-dimethyl-1,3-propanediol, glycerol, or propylene glycol might be advantageous in specific instances.

### DESCRIPTION OF PREFERRED EMBODIMENT

Since amine nucleophilicity/steric accessibility and ester electrophilicity/steric accessibility vary widely, it is not possible to recommend a particular set of reaction conditions that will be applicable to all conceivable ester-amine combinations. Instead it was found convenient to categorize esters as possessing high, medium, or low “electrophilicity-steric accessibility” (i.e. intrinsic reactivity toward an “average amine”), and to categorize amines as having high, medium or low “nucleophilicity-steric accessibility” (i.e. intrinsic reactivity toward an “average ester”), thereby obtaining a 3×3 “intrinsic reactivity matrix”.

On the basis of this matrix and of our experimental results, we have ranked the reactivity of different ester-amine pairs as follows.

| | INTRINSIC | INTRINSIC | INTRINSIC |
|---|---|---|---|
| REACTIVITY | ESTER | AMINE | COMPARATIVE |
| GROUP | REACTIVITY | REACTIVITY | REACTIVITY |
| I | HIGH | HIGH | +++++ |
| II | HIGH | MEDIUM | ++++ |
| | MEDIUM | HIGH | |
| III | HIGH | LOW | |
| | MEDIUM | MEDIUM | +++ |
| | LOW | HIGH | |
| IV | MEDIUM | LOW | ++ |
| | LOW | MEDIUM | |
| V | LOW | LOW | + |

SYMBOLOGY:
+++++ VERY HIGH COMPARATIVE REACTIVITY
++++ HIGH COMPARATIVE REACTIVITY
+++ MODERATE COMPARATIVE REACTIVITY
++ LOW COMPARATIVE REACTIVITY
+ VERY LOW COMPARATIVE REACTIVITY

Therefore, five “reactivity groups” emerged, and it was possible to establish the preferred embodiment for each group.

Before describing these 5 sets of conditions, it is important to identify the specific types of esters and amines that belong in each “intrinsic reactivity” category. High-reactivity esters.: formates, oxalates, carbonates, fumarates, aromatic esters (benzoates, naphthoates, etc.) bearing electron-withdrawing groups, heteroaromatic esters (furoates, pyridinecarboxylates, etc.)

Medium-reactivity esters: sterically unhindered esters derived from saturated aliphatic carboxylic acids, benzoates, naphthoates, oxamates, crotonates, and cinnamates.

Low-reactivity esters: sterically hindered esters derived from saturated, unsaturated or aromatic carboxylic acids, aromatic esters (benzoates, naphthoates, etc.) bearing electron-releasing substituents, heteroaromatic esters bearing electron-releasing groups, carbamates.

High-reactivity amines: primary aliphatic amines devoid of steric hindrance, dimethylamine, monoethanolamine, morpholine, pyrrolidine, piperidine, primary aromatic amines bearing strongly electron-releasing groups, hydrazine.

Medium-reactivity amines: secondary aliphatic amines (excepting dimethylamine), aniline, alpha-naphthylamine, beta-naphthylamine, primary aromatic amines bearing moderately electron-releasing substituents, ammonia, aminoacids salts.

Low-reactivity amines: highly hindered primary aliphatic, secondary aliphatic and primary aromatic amines, secondary aliphatic-aromatic amines, secondary aromatic amines, heteroacyclic amines, primary aromatic amines bearing electron-withdrawing substituents.

Preferred embodiments for the synthesis of amides belonging to each of the five “reactivity groups” are as follows.

Group I Amides

Equimolar amounts of dry ethyl or (most preferably) methyl ester, dry amine and >99% pure or (most preferably) anhydrous ethylene glycol are admixed, the reaction mixture is heated at reflux temperature until the reaction is complete as evidenced by disappearance of the ester or amine (T.L.C) and the amide is separated by means that are contingent upon its physical properties.

NOTE: If a diester is used, 2 moles of amine per mol of ester should be employed. If the diamine is used, 2 moles of ester per mole of amine should be employed.

Group II Amides

The procedure is similar to the one outlined for group I amides, except for the use of a molar ratio Glycol:ester:amine=4-10:1:1 and (most preferably) the addition of a catalytic amount of sodium methoxide

Group III Amides

In general, the procedure is similar to the one outlined for “group II amides”, except for the use of a stoichiometric amount of sodium methoxide

Group IV Amides

The procedure is similar to the one just given for synthesizing “group III amides”, but either superatmospheric pressures should be applied or the alcohol should be removed from the reaction medium as it is formed (Dean-Stark trap)

Group V Amides

The general synthetic protocol is similar to that given above for “group IV amides” but higher pressures and/or temperatures must be applied.

### EXAMPLES

The following non-limiting examples are intended to promote a further understanding of the present invention.

### Utility Example 1

Nicotinamide

Starting Materials

| Ethyl Nicotinate | 0.5 | mol |
|---|---|---|
| Ammonia (gas) | excess, bubbled through system | |
| Ethylenglycol | 120 | ml |

Operating Conditions

| Pressure | Atmospheric | |
|---|---|---|
| Temperature/time regime | 40-50° | C./6 h. |
| Reaction Progress | | |
| Monitored by TLC | | |
| Work-up | | |

1 liter of water added, the solution extracted with chloroform (5 ×
100 mL); the organic phase separated, chloroform eliminated by
distillation at atmospheric pressure and the solid residue recrystallized
from benzene. 5 g. of crystals were obtained, m.p. 128.5-129.5° C.
(Literature 130° C.)

| | |
|---|---|
| Yield | 8% |

### Comparative Example 1

Nicotinamide

| Starting Materials | | |
|---|---|---|
| Ethyl Nicotinate | 0.5 | mol |
| Ammonia (gas) | Excess, bubbled through system | |
| Ethanol | 100 | mL |
| Sodium methoxide (catalyst) | 3.0 | g. |
| Operating Conditions | | |
| Pressure | Atmospheric | |
| Temperature/time regime | 55° | C./26 h. |
| Reaction Progress | | |
| Monitored by TLC | | |
| Yield | 0% | |

### Utility Example 2

3-Nitrobenzamide

Starting Materials

| Methyl 3-Nitrobenzoate | 0.15 | mol |
|---|---|---|
| Ammonia (gas) | Excess, bubbled through system | |
| Ethylenglycol | 125 | mL |
| Sodium methoxide (catalyst) | 0.09 | mol |

Operating Conditions

| Pressure | Atmospheric | |
|---|---|---|
| Temperature/time regime | 40-45° | C./20 h.; |
| | then 40-45° | C./\hat{ }5 h. |
| | (after adding the catalyst) | |
| Reaction Progress | | |
| Monitored by TLC | | |
| Work-up | | |

The reaction mixture was heated to 100° C., mixed with 500 mL
water, heated to 80° C., filtered while hot (to remove insoluble matter),
cooled to 10° C., filtered to separate the precipitate, the crystals washed
with cold water (100 mL) and dried at 70-80° C./12 h. 20 g. of yellowish
crystals were obtained m.p. 143.3-144.1° C. (literature m.p. 143° C.). A
second crop (3 g.) of crystals was obtained from the cooled mother liquor.

| | |
|---|---|
| Yield | 92% |

### Comparative Example 2

3-Nitrobenzamide

Starting Materials

| Methyl 3-Nitrobenoate | 0.15 | mol |
|---|---|---|
| Ammonia (gas) | Excess, bubbled through system | |
| Methanol (anhydrous) | 100 | mL |
| Sodium methoxide (catalyst) | 8 | g. |

Operating Conditions

| Pressure | Atmospheric | |
|---|---|---|
| Temperature/time regime | 60-65° | C./26 h. |
| Reaction Progress | | |
| Monitored by TLC | | |
| Work-up | | |

Most of the methanol was eliminated by distillation at atmospheric
pressure. 500 mL of water were added and the mixture was cooled to room
temperature. The precipitate was separated by filtration under reduced
pressure, washed with water and dried at 70-75° C. during 12 hours. 17
g. of cream-colored crystals were obtained, m.p. 142.7-143.3° C.

| | |
|---|---|
| Yield | 68% |

### Comparative Example 3

3-Nitrobenzamide

Starting Materials

| Methyl 3-Nitrobenoate | 0.057 | mol |
|---|---|---|
| Ammonia (gas) | Excess, bubbled through system | |
| n-Butanol | 180 | mL |
| Sodium methoxide (catalyst) | 3 | g. |

Operating Conditions

| Pressure | Atmospheric | |
|---|---|---|
| Temperature/time regime | 40-45° | C./20 h.; then |
| | 40-45° | C./8 h. |
| | (after adding the catalyst | |
| Reaction Progress | | |
| Monitored by TLC | | |
| Work-up | | |

The solvent was eliminated by adding water (50 mL) and distilling
the azeotrope at atmospheric pressure with further addition of
water so as to keep a constant volume. The product was extracted
with chloroform and the extract subjected to distillation at
atmospheric pressure in order to eliminate the chloroform. Only 6
g. of solid residue were recovered, most of which was shown by
TLC to be Methyl 3-Nitrobenzoate.

| | |
|---|---|
| Yield | Nil. |

### Utility Example 3

N,N,N′,N′-tetramethylterephthaldiamide (method 1)

Starting Materials

| | | |
|---|---|---|
| Dimethyl terephthalate | 0.5 | mol |
| Anhydrous dimethylamine | 1.1 | mol |
| Ethylene Glycol | 400 | g. |

Operating Conditions

| | | |
|---|---|---|
| Pressure | 50 | psi |
| Temperature/time regime | 90-100° | C./8 h |

Reaction Progress
Monitored by TLC: Kodak's silica gel plates with flourescent
indicator; benzene-acetone-ethyl acetate (34.5:61.5:4).
Work-up
The reaction mass was allowed to cool, mixed with saturated
aqueous sodium chloride solution and exhaustively extracted with
chloroform. The solvent was evaporated from the combined extracts and
the residue was recrystallized in benzene and dried overnight at 70-80° C.

| | |
|---|---|
| Yield | 45% |

### Utility Example 4

N,N,N′,N′-tetramethylterephthaldiamide (method 2)

Starting Materials

| | | |
|---|---|---|
| Dimethyl terephthalate | 0.5 | mol |
| Anhydrous dimethylamine | 3.0 | mol |
| Ethylene Glycol | 400 | mL |

Operating Conditions

| | | |
|---|---|---|
| Pressure | 70 | psi |
| Temperature/time regime | 90-100° | C./7 h |
| Reaction Progress | | |
| Not monitored. | | |
| Work-up | | |

The reaction mass was allowed to cool, mixed with saturated
aqueous sodium chloride solution and exhaustively extracted with
chloroform. The pooled extracts were distilled to eliminate chloroform and
the residue was dried overnight at 70-80° C. The dry product melted at
183-190° C. (literature m.p. 200-201° C.). The crude product was light
brown, after recrystallization from 96% ethanol, it melted at 200-201° C.
(white crystals). This product was characterized by IR and by
determination of nitrogen content (Kjeldahl).

| | | |
|---|---|---|
| Yield | 89.5% | (crude) |

### Utility Example 5

N,N,N′,N′-tetramethylterephthaldiamide (method 3)
Starting Materials

| | | |
|---|---|---|
| Dimethyl terephthalate | 1.0 | mol |
| Anhydrous dimethylamine | 6.0 | mol |
| Ethylene Glycol | 800 | mL |

Operating Conditions

| | | |
|---|---|---|
| Pressure | 60 | psi |
| Temperature/time regime | 92-108° | C./6 h. |
| Reaction Progress | | |
| Not monitored. | | |
| Work-up | | |

The reaction mass was allowed to cool, mixed with saturated
aqueous sodium chloride solution and exhaustively extracted with
chloroform. The pooled extracts were distilled to eliminate chloro-
form and the residue was dried overnight at 70-80° C., melting at
187-190° C. (literature m.p. 200-201° C.). Its purity was 98.3%
(HPLC) and its identity was confirmed by IR and by determination of
nitrogen content (Kjeldahl).

| | |
|---|---|
| Yield (Crude) | 91% |
| Yield (Recrystallized from 96% ethanol) | 68% |

### Utility Example 6

N,N,N′,N′-tetramethylterephthaldiamide (method 4)

Starting Materials

| | | |
|---|---|---|
| Dimethyl terephthalate | 1.0 | mol |
| Anhydrous dimethylamine | 6.0 | mol |
| Ethylene Glycol | 800 | mL |

Operating Conditions

| | | |
|---|---|---|
| Pressure | 5 | psi |
| Temperature/time regime | 59-63° | C./22.5 h |
| Reaction Progress | | |
| Monitored by HPLC | | |
| Work-up | | |

Unreacted dimethylamine and by-product methanol were
eliminated by distillation at atmospheric pressure. The residue was cooled
at 0-5° C., and filtered, the filter cake washed with cold acetone, drained
and dried overnight at 100° C. 138.5 g. of crystals were obtained, with a
purity of 98.8% (HPLC). The filtrate (1063 g.) contained 4.2% (w/w)
tetramethylterephthaldiamide (44.7 g.)

| | |
|---|---|
| Yield (Total) | 83% |
| Yield (Isolated) | 63% |

### Utility Example 7

N,N,N′,N′-tetramethylterephthaldiamide (method 5)

Starting Materials

| | | |
|---|---|---|
| Dimethyl terephthalate | 1.726 | mol |
| Anhydrous dimethylamine | 6 | mol |
| Ethylene Glycol | 685 | mL |

Operating Conditions

| | | |
|---|---|---|
| Pressure | 35 | psi |
| Temperature/time regime | 58-62° | C./13 h |
| Reaction Progress | | |
| Not monitored. | | |
| Work-up | | |

The reaction mass was allowed to cool, kept for a couple of hours
ay 0-5° C. and filtered. The filter cake was washed with cold acetone,
drained and dried overnight at 70-80° C. 266.3 g. of cream-colored
crystals were obtained.

| | | |
|---|---|---|
| Yield | 70.1% | (isolated) |

### Utility Example 8

N,N,N′,N′-tetramethylterephthaldiamide (method 6)

Starting Materials

| | | |
|---|---|---|
| Dimethyl terephthalate | 3 × 1.185 | mol |
| Anhydrous dimethylamine | 3 × 6 | mol |
| Ethylene Glycol | 1 | mL |

Operating Conditions

| | | |
|---|---|---|
| Pressure | 10-11 | psi (first cycle) |
| | 8-13 | psi (second cycle) |
| | 7-10 | psi (third cycle) |
| Temperature/time regime | 58-63° | C./12 h 10 min (first cycle) |
| | 59-68° | C./11 h (second cycle) |
| | 60-62° | C./18 h 15 min (third cycle) |
| Reaction Progress | | |
| Monitored by HPLC. | | |
| Work-up | | |

At the end of each cycle the reaction mixture was distilled at
atmospheric pressure in order to eliminate the excess dimethylamine and
by-product methanol. The residue was subjected to “clarification” by
treating it with activated charcoal and celite while hot and then fil-
tering. The filtrate was cooled at 0-5° C. and stirred during several
hours, the precipitate separated by filtration, washed with cold ace-
tone or cold isopropyl alcohol, drained and the filter cake dried over-
night at 80-100° C.
Yield
First cycle gave an isolated yield of 67.3%
Second cycle gave an isolated yield of 67.5%
Third cycle gave an isolated yield of 84.5%
Global yield 81.3%
This product was 99.6% pure by HPLC.

### Comparative Example 4

N,N,N′,N′-tetramethylterephthaldiamide

| Starting Materials | | |
|---|---|---|
| Dimethyl terephthalate | 1.0 | mol |
| Anhydrous dimethylamine | 6.0 | mol |
| N,N-dimethylformamide | 800 | mL |
| Operating Conditions | | |
| Pressure | 120 | psi |
| Temperature/time regime | 93-101° | C./5.3 h.; then |
| | 98-102° | C./8.4 h. |
| Reaction Progress | | |
| Not monitored | | |
| Yield | Less than 5% | |

### Comparative Example 5

N,N,N′,N′-tetramethylterephthaldiamide

Starting Materials

| | | |
|---|---|---|
| Dimethyl terephthalate | 1.0 | mol |
| Anhydrous dimethylamine | 10.3 | mol |
| Methanol | 300 | mL |

Operating Conditions

| | | |
|---|---|---|
| Pressure | 140 | psi |
| Temperature/time regime | 88-100° | C./5.5 h |
| Reaction Progress | | |
| Not monitored | | |
| Work-up | | |

The reactions mixture was allowed to cool, diluted with water,
saturated with sodium chloride, heated at 70-80° C. over 30 minutes,
cooled and exhaustively extracted with chloroform, the solvent evaporated
form the combined extracts and the residue dried overnight at 70-80° C.

| | |
|---|---|
| Yield | 22% |

### Comparative Example 6

N,N,N′,N′-tetramethylterephthaldiamide

Starting Materials

| | | |
|---|---|---|
| Dimethyl terephthalate | 0.5 | mol |
| Anhydrous dimethylamine, | 60% | w/w 5 mol |

Operating Conditions

| Pressure | Atmosphere | |
|---|---|---|
| Temperature/time regime | Reflux/18 | h. |
| Reaction Progress | | |

Monitored by TLC: Kodak silica gel TLC plates with fluorescent
indicator, benzene-acetone-ethylacetate (34.5:61.5:4.0)
Work-up
Most of the excess dimethylamine was removed by heating,
sodium chloride was added until a saturated solution was obtained.
The product was extracted exhaustively with chloroform, the
chloroform eliminated from the extract by evaporation and the residue
dried overnight at 70-80 An off-white solid were obtained (m.p. 197-
198° C.) (literature m.p. 200-201° C.)

| | |
|---|---|
| Yield | 45.5% |

### Comparative Example 7

N,N,N′,N′-tetramethylterephthaldiamide

Starting Materials

| | | |
|---|---|---|
| Dimethyl terephthalate | 0.5 | mol |
| Anhydrous dimethylamine | 11.1 | mol |

Operating Conditions

| | | |
|---|---|---|
| Pressure | 125 | psi |
| Temperature/time regime | 64-70° | C./9 h. |
| Reaction Progress | | |
| Not monitored | | |
| Work-up | | |

The reaction mixture was quenched with water, excess
dimethylamine evaporated by heating at 70-80° C. during 1 h, and
the product separated by exhaustive chloroform extraction. The
chloroform was eliminated by distillation from the extract and
the residue was dried overnight at 70-75° C.
Note: an insoluble solid by-product was separated form the
reaction mixture. Its properties matched those of 4-carboxy-N,N-
dimethylbenzamide

| | |
|---|---|
| Yield | 44.5% |

### Utility Example 9

2-Furancarboxamide

Starting Materials

| Ethyl-2-Furancarboxylate | 0.5 | mol |
|---|---|---|
| Ammonia (gas) | Excess, bubbled through system | |
| Ethylene glycol | 150 | g. |

Operating Conditions

| Pressure | Atmospheric | |
|---|---|---|
| Temperature/time regime | 48-49° | C./12 h. |
| Work-up | | |

One liter of water was added, pH was adjusted to 6.5-7.0 with 10%
aqueous HCl, the aqueous solution was extracted with chloroform (5 ×
100 mL), the organic phase was separated and the chloroform eliminated
by distillation at atmospheric pressure leaving 15 g. of a cream-colored
residue, m.p. 141.8-142.7° C.
The aqueous mother liquor was concentrated to 600 mL by
evaporation, saturated with sodium chloride, extracted with chloroform
(5 × 100 mL) and the extract treated as above, yielding another crop
of cream-colored crystals. (15 g, m.p. 141.7-142.8° C.)

| | |
|---|---|
| Yield | 51% |

### Utility Example 10

1-Naphthalenecarboxamide

Starting Materials

| Ethyl-1-Naphthalenecarboxylate | 0.1 | mol |
|---|---|---|
| Ammonia (gas) | Excess, bubbled through system | |
| Ethylene glycol | 100 | g. |
| Sodium methoxide (catalyst) | 3.0 | g. |

Operating Conditions

| Pressure | Atmospheric | |
|---|---|---|
| Temperature/time regime | 35° | C./20 h.; then |
| | 70-75° | C./8 h. |
| Reaction Progress | | |
| Monitored by TLC | | |
| Work-up | | |

The reaction mixture was allowed to cool to room temperature,
1200 mL of water added, the solution's pH adjusted to 6.5 using 10%
aqueous HCl, and allowed to cool at room temperature. The solid that
precipitated was separated by filtration under reduced pressure, washed
with cold water, dispersed into 300 mL anhydrous ethanol, recovered by
filtration under reduced pressure and dried. 6.0 g. of yellowish powdery
crystals were obtained, m.p. 206-206.8° C. (literature 202° C.)

| | |
|---|---|
| Yield | 35% |

### Utility Example 11

N,N-Dimethylbenzamide

Starting Materials

| | | |
|---|---|---|
| Methyl benzoate | 0.5 | mol |
| DIM-CARB | 2.5 | mol |
| (Dimethylammonium | | |
| N,N-Dimethylcarbamate) | | |
| Ethylene glycol | 200 | mL |
| Tetra iso-propyl | 0.1 | mol |
| titanate (catalyst) | | |

Operating Conditions

| Pressure | Atmospheric | |
|---|---|---|
| Temperature/time regime | 60-65° | C./6 h.; then |
| | 72-75° | C./24 h. |
| Reaction Progress | | |
| Monitored by TLC | | |
| Work-up | | |

The unreacted DIM-CARB was eliminated by distillation at
atmospheric pressure, 1500 ml water added, pH adjusted to 6.5 with 10%
aqueous HCl and the solution thoroughly extracted with chloroform.
Chloroform was eliminated from extract by distillation at atmospheric
pressure, and the residue distilled under reduced pressure yielding 29.5
g. of pure product (only one spot by thin layer chromatography)

| | |
|---|---|
| Yield | 40% |

### Utility Example 12

Terephthaldiamide (Method 1)

Starting Materials

| Dimethyl Terephthalate | 0.1 | mol |
|---|---|---|
| Ammonia (gas) | Excess, bubbled through system | |
| Magnesium Methoxide (catalyst) | 0.027 | mol |
| Ethylene Glycol | 300 | mL |

Operating Conditions

| Pressure | Atmospheric | |
|---|---|---|
| Temperature/time regime | Room temperature/1 | h.; |
| | then 80° | C./24 h. |
| Reaction Progress | | |
| Monitored by TLC | | |
| Work-up | | |

The reaction mixture was cooled to room temperature, 1800 mL
water added, pH adjusted to 3 with 50% aqueous sulfuric acid. The
system was heated to 70-80° C. and maintained at that temperature during
30 minutes, cooled to room temperature and filtered under reduced
pressure. The filter cake was washed thoroughly with water, drained and
dried at 70-75° C. overnight. 16.3 g. of white, powdery crystals were
obtained, m.p. 322.3-323.8° C. (Literature 330° C.)

| | |
|---|---|
| Yield | 99% |

### Utility Example 13

Terephthaldiamide (Method 2)

Starting Materials

| | | |
|---|---|---|
| Dimethyl Terephthalate | 0.1 | mol |
| Ammonium Carbonate | 2.0 | mol |
| Ethylene Glycol | 350 | mL |

Operating Conditions

| Pressure | Atmospheric | |
|---|---|---|
| Temperature/time regime | 75-80° | C./40 h. |
| Reaction Progress | | |
| Monitored by TLC | | |
| Work-up | | |

The reaction mixture was cooled to 20° C., 1.5 L water added, pH
adjusted to 6 with concentrated aqueous hydrochloric acid, cooled to 20°
C. and filtered under reduced pressure. The filter cake was washed with
200 mL water, dried at 70-80° C. overnight, dispersed in 1 L methanol
(absolute) at 70-75° C. during 30 minutes, and filtered under reduced
pressure. 5.8 g. of white crystals were obtained (only one spot was
observed by TLC)

| | |
|---|---|
| Yield | 35% |

### Utility Example 14

Terephthaldiamide (Method 3)

Starting Materials

| | | |
|---|---|---|
| Dimethyl terephthalate | 0.1 | mol |
| Urea | 4.0 | mol |
| Ethylene Glycol | 350 | mL |

Operating Conditions

| Pressure | Atmospheric | |
|---|---|---|
| Temperature/time regime | 120-125° | C./30 h. |
| Reaction Progress | | |
| Monitored by TLC | | |
| Work-up | | |

The reaction mixture was cooled to room temperature, 1 L water
added, the pH adjusted to 7 with concentrated aqueous hydrochloric acid,
heated to 80-85° C. and maintained at this temperature during 30 minutes.
Then it was slowly cooled to room temperature, filtered under reduced
pressure; the filter cake was washed with 200 mL cold water, drained, and
dried at 70-80° C. during 24 h. 12.0 g. of white powdery crystals were
obtained, m.p. 329.3-330.8° C. (literature m.p. 330° C.)

| | |
|---|---|
| Yield | 73% |

### Utility Example 15

Barbituric acid

Starting Materials

| | | |
|---|---|---|
| Diethyl malonate | 0.5 | mol |
| Urea | 0.55 | mol |
| Sodium methoxide (catalyst) | 0.5 | mol |
| Ethylene Glycol | 223 | g. |

Operating Conditions

| Pressure | Atmospheric | |
|---|---|---|
| Temperature/time regime | 110° | C./6 h. |
| Reaction Progress | | |

Monitored by TLC using Merck's silica gel plates, benzene-
methanol (1:1)

Work-up

The reaction mixture was cooled to 60° C., diluted with 500 mL
water at 50° C., made acidic (to a blue color with congo red indicator)
by addition of 55.8 g. concentrated aqueous hydrochloric acid, refri-
gerated overnight and filtered. The filter cake was washed with 50 mL
cold (10° C.) water and dried at 90° C. during 4 h. 44.6 g, of white
crystals, m.p. 245° C. (dec.) were obtained (literature 248° C. “with
some decomposition”)

| | |
|---|---|
| Yield | 70% |

### Utility Example 16

N,N′-bis(4-hydroxyphenyl) oxamide (Method 1)

Starting Materials

| | | |
|---|---|---|
| N-(4-hydroxyphenyl) oxamate | 0.25 | mol |
| p-Aminophenol | 0.25 | mol |
| Ethylene Glycol | 150 | g. |

Operating Conditions

| Pressure | Atmospheric | |
|---|---|---|
| Temperature/time regime | 80° | C./5 h.; then |
| | 100° | C./1 h. |
| Reaction Progress | | |

Monitored by TLC using Merck's silica gel plates, benzene-acetone
(3:1).

Work-up

The reaction mixture was allowed to cool, quenched with 800 mL
water and filtered. The filter cake was washed with 200 mL water, then
with 500 mL cold acetone, drained and dried overnight at 80° C. 52.0 g.
of product were obtained, m.p. 350° C. (dec)

| | |
|---|---|
| Yield | 76% |

### Utility Example 17

N,N′-bis(4-hydroxyphenyl) oxamide (Method 2)

Starting Materials

| | | |
|---|---|---|
| Diethyl oxalate | 0.15 | mol |
| p-Aminophenol | 0.30 | mol |
| Ethylene Glycol | 111 | g. |

Operating Conditions

| Pressure | Atmospheric | |
|---|---|---|
| Temperature/time regime | 90° | C./4 h. |
| Reaction Progress | | |

Monitored by TLC using Merck's silica gel plates, benzene-acetone
(3:1).

Work-up

The reaction mixture was allowed to cool, quenched with 400 mL
water and filtered. The filter cake was washed with water, then with
300 mL cold acetone, drained and dried overnight at 80° C. 25.8 g.
of off-white crystals were obtained, m.p. 350° C. (dec)

| | |
|---|---|
| Yield | 96% |

### Utility Example 18

N-(4-hydroxyphenyl) oxamic acid, 2-hydroxyethyl ester

Starting Materials

| | | |
|---|---|---|
| Diethyl oxalate | 0.9 | mol |
| p-Aminophenol | 0.3 | mol |
| Ethylene Glycol | 446 | g. |

Operating Conditions

| Pressure | Atmospheric | |
|---|---|---|
| Temperature/time regime | 80-85° | C./4 h. |
| Reaction Progress | | |

Monitored by TLC using Merck's silica gel plates, benzene-acetone
(3:1).

Work-up

The reaction mixture was allowed to cool, extracted with 2 × 200
mL diethyl ether, diluted to a total volume of 3 L with water and stored
at room temperarute for 2 h. Later it was filtered, the filter cake
drained and dried at 80° C. overnight. 26.3 g. of purple crystals (m.p.
160-162° C.) were obtained

| | |
|---|---|
| Yield | 39% |

### Utility Example 19

N, N′-Diphenyloxamide

Starting Materials

| | | |
|---|---|---|
| Diethyl oxalate | 0.11 | mol |
| Aniline | 0.88 | mol |
| Ethylene Glycol | 150 | g. |

Operating Conditions

| Pressure | Atmospheric | |
|---|---|---|
| Temperature/time regime | 120-125° | C./6 h. |
| Reaction Progress | | |

Monitored by observing changes in reaction mass.

Work-up

The reaction mixture was cooled to 5-10° C., and filtered. Later the
filter cake was drained, dispersed in 100 mL cold ethanol, filtered,
the filter cacke washed with 100 mL cold ethanol, drained, and dried
at 70-80° C. overnight. 25.4 g. of yellowish crystals, m.p. 252-253° C.
were obtained (lit m.p. 252-254° C.)

| | |
|---|---|
| Yield | 96% |

### Utility Example 20

N-(2-hydroxyethyl)-N′-(4-hydroxyphenyl) oxamide

Starting Materials

| | | |
|---|---|---|
| Ethyl N-(4-hydroxyphenyl) oxamate | 0.25 | mol |
| Monoethanolamine | 0.25 | mol |
| Ethylene Glycol | 150 | g. |

Operating Conditions

| Pressure | Atmospheric | |
|---|---|---|
| Temperature/time regime | 80-85° | C./1 h. |
| Reaction Progress | | |

Monitored by TLC using Merck's silica gel plates, benzene-
dimethylformamide (25:4).

Work-up

The reaction mass was allowed to cool to room temperature (20
° C.) Later it was filtered, the filter cake drained, washed with cold water,
drained again and dried at 80° C. overnight. 51.8 g. of off-white crystals
(m.p. 234-235° C.) were obtained

| | |
|---|---|
| Yield | 93% |

### Utility Example 21

N,N′-bis (2-hydroxyethyl) oxamide

Starting Materials

| | | |
|---|---|---|
| Diethyl oxalate | 0.25 | mol |
| Monoethanolamine | 0.5 | mol |
| Ethylene Glycol | 250 | g. |

Operating Conditions

| Pressure | Atmospheric | |
|---|---|---|
| Temperature/time regime | 79-80° | C./3 h. |
| Reaction Progress | | |

Monitored by TLC using Merck's silica gel plates and benzene-
dimethylformamide (25:4).

Work-up

The reaction mass was allowed to cool to room temperature,
refrigerated (0° C.) and stirred for five minutes before filtration. The filter
cake was drained, dried at 70-80° C. overnight, dispersed in ethanol (3
parts ethanol to 1 part solid), the dispersion heated to boiling, cooled and
filtered. The filter cake was washed with cold ethanol, drained and dried
overnight at 60-80° C. 38.2 g. of white crystals (m.p. 169.9-170.3° C.)
were obtained (literature m.p. 166-169° C.)

| | |
|---|---|
| Yield | 87% |

### Utility Example 22

p-acetamidobenzoic acid

Starting Materials

| | | |
|---|---|---|
| Ethyl Acetate | 0.75 | mol |
| Sodium p-aminobenzoate | 0.25 | mol |
| Sodium methoxide (catalyst) | 0.25 | mol |
| Ethylene Glycol | 150 | g. |

Operating Conditions

| Pressure | Atmospheric |
|---|---|
| Temperature/time regime | 75° C./8 h.; then 90° C./2 h.; |
| | then 110° C./9 h.; then 120-125° |
| | C./2.5 h.; then 135° C./4.5 h. |
| Reaction Progress | |
| Monitored by TLC | |
| Work-up | |

The reaction mass was allowed to cool to 40° C., transferred to a
beaker, diluted with water to a total volume of 500 mL, pH adjusted to
2-3 by addition of 51.3 g. concentrated hydrochloric acid, cooled to
10° C., stirred during 30 min at that temperature and filtered. The
filter cake was later drained, washed with 100 mL cold water, drained
thoroughly and dried at 60-68° C. to constant weight. 21.5 g. of crys-
tals (m.p. 258-259° C. (dec.))were obtained (literature 252° C.)

| | |
|---|---|
| Yield | 48% |

### Utility Example 23

Phenacetin (method 1)

Starting Materials

| | | |
|---|---|---|
| Ethyl Acetate | 0.44 | mol |
| p-phenetidine | 0.3 | mol |
| Sodium methoxide (catalyst) | 0.1 | mol |
| Ethylene Glycol | 150 | g. |

Operating Conditions

| Pressure | Atmospheric | |
|---|---|---|
| Temperature/time regime | 106-110° | C./3 h; |
| | then 130° | C./7 h. |
| Reaction Progress | | |
| Monitored by TLC | | |
| Work-up | | |

The reaction mass was allowed to cool to room temperature,
diluted with 250 mL water, stirred to control crystal size and filtered.
The filter cake was then washed with 50 mL water, drained and dried to
constant weight at 70-80° C. 31.6 g. of dark brown crystals were
obtained, melting at 133.9-135.1° C. (literature 134-135° C.).

| | |
|---|---|
| Yield | 59% |

### Utility Example 24

Phenacetin (method 2)

Starting Materials

| | | |
|---|---|---|
| Ethylene glycol diacetate | 0.25 | mol |
| p-phenetidine | 0.25 | mol |
| Sodium methoxide (catalyst) | 0.1 | mol |
| Ethylene Glycol | 150 | g. |

Operating Conditions

| Pressure | Atmospheric | |
|---|---|---|
| Temperature/time regime | 120-125° | C./3 h |
| Reaction Progress | | |
| Monitored by TLC | | |
| Work-up | | |

The reaction mass was allowed to cool to room temperature,
diluted with 200 mL water, stirred to control crystal size and filtered.
The filter cake was then washed with 50 mL water, drained and dried to
constant weight at 70-80° C. 33.4 g. of dark brown crystals were
obtained, melting at 134-135.2° C.

| | |
|---|---|
| Yield | 75% |

### Utility Example 25

Phenacetin (method 3)

Starting Materials

| | | |
|---|---|---|
| Triacetin | 0.2 | mol |
| p-phenetidine | 0.2 | mol |
| Sodium methoxide (catalyst) | 0.1 | mol |
| Ethylene Glycol | 150 | g. |

Operating Conditions

| Pressure | Atmospheric | |
|---|---|---|
| Temperature/time regime | 120-125° | C./3 h |
| Reaction Progress | | |
| Monitored by TLC | | |
| Work-up | | |

The reaction mass was allowed to cool to room temperature,
diluted with 200 mL water, stirred for 30 minutes to control crystal
size and filtered. The filter cake was then washed with 200 mL water,
drained thoroughly and dried to constant weight at 70-80° C. 28.3 g.
of dark brown crystals were obtained, melting at 134-135.5° C.

| | |
|---|---|
| Yield | 79% |

### Utility Example 26

N-cyclohexylbenzamide

Starting Materials

| | | |
|---|---|---|
| Ethyl benzoate | 0.5 | mol (75 g) |
| Cyclohexyylamine | 0.6 | mol (59.5 g) |
| Sodium methoxide (catalyst) | 10 | g. |
| Ethylene Glycol | 50 | g. |

Operating Conditions

| Pressure | Atmospheric | |
|---|---|---|
| Temperature/time regime | 120° | C./7 h; then |
| | 125° | C./6 h. |
| Reaction Progress | | |
| Monitored by TLC | | |
| Work-up | | |

The reaction mass was allowed to cool to room temperature,
transferred to a beaker, diluted with 700 mL methanol and 300 mL water,
its pH adjusted to 7 using a few milliliters of concentrated aqueous
hydrochloric acid, stirred for one hour and filtered. The filter cake was
then washed with water, drained and dried at 70-80° C. overnight. 32.5 g.
of white crystals (m.p. 149.3-150.7° C.) were obtained (lit m.p. 148-149
° C.)

| | |
|---|---|
| Yield | 32% |

### Utility Example 27

Palmitamide

Starting Materials

| Methyl palmitate | 0.5 | mol (135 g) |
|---|---|---|
| Ammonia | Excess, bubbled through the | |
| | system. | |
| Sodium methoxide (catalyst) | 5 | g. |
| Ethylene Glycol | 50 | g. |

Operating Conditions

| Pressure | Atmospheric | |
|---|---|---|
| Temperature/time regime | 65° | C./8 h. |

Reaction Progress
Monitored by means of the qualitative ferric hydroxamate test for
esters.
Work-up
The reaction mass was cooled, transfered to a beaker, diluted with
500 mL methanol and 200 mL water, stirred for 30 minutes and
filtered. The filter cake was then washed with water, drained and
dried at 70-80° C. overnight. 116 g. of white powdery crystals
(m.p. 102-103° C.) were obtained (literature m.p. 106-107° C.);
the IR spectrum (KBr pellet) shows the “amide I Band” at 1647.42
CM⁻¹ and the “C—N Stretch” at 1421.72 CM⁻¹.

| | |
|---|---|
| Yield | 91% |

### Utility Example 28

N,N-Diethylnicotinamide (Nikethamide)

Starting Materials

| | | |
|---|---|---|
| Ethyl nicotinate | 0.5 | mol |
| Diethylamine | 1.5 | mol |
| Sodium methoxide (catalyst) | 4.9 | g. |
| Ethylene Glycol | 150 | g. |

Operating Conditions

| Pressure | Atmospheric | |
|---|---|---|
| Temperature/time regime | 85° | C./25 h. |
| Reaction Progress | | |
| Monitored by TLC | | |
| Work-up | | |

The unreacted diethylamine was separated by distillation of the
reaction mass at atmospheric pressure (70 mL of liquid were collected),
the residue was transferred to a beaker, diluted with water to a total
volume of 1200 mL, extracted with 5 × 100 mL chloroform and the
combined organic phases distilled at atmospheric pressure to eliminate
the solvent. The residue weighed 33.3 g.; its purity was found to be 98%
by perchloric acid titration in glacial acetic acid.

| | |
|---|---|
| Yield | 37% |

### Utility Example 29

m-chloroformanilide

Starting Materials

| | | |
|---|---|---|
| Ethyl formate | 1.5 | mol (111 g) |
| m-chloraniline | 0.5 | mol (64 g) |
| Sodium methoxide (catalyst) | 10 | g. |
| Ethylene Glycol | 50 | g. |

Operating Conditions

| Pressure | Atmospheric | |
|---|---|---|
| Temperature/time regime | 65-72° | C./24 h. |
| Reaction Progress | | |

Monitored by TLC: Merck's silica gel plates; benzene-methanol
(20:3)
Work-up
The unreacted ester and the by-product ethanol (60 mL) were
removed from the reaction mass by distillation at atmospheric pressure.
The residue was allowed to cool, quenched with 200 mL water, its pH
adjusted to 6 by adding concentrated aqueous hydrochloric acid (2-3 mL)
and the mixture heated during 30 minutes. A biphasic system was
obtained, the phases separated in a funnel and the organic (lower) phase
was washed with 300 mL water and then cooled, yielding crystals that
weighed 61.5 g and melted at 57-58.5° C. (literature 57-58° C.).

| | |
|---|---|
| Yield | 79% |

### Utility Example 30

Ethyleneurea

Starting Materials

| | | |
|---|---|---|
| Urea | 0.25 | mol (15 g) |
| Ethylenediamine | 0.25 | mol (15 g) |
| Ethylene Glycol | 1.45 | mol (90 g) |

Operating Conditions

| Pressure | Atmospheric | |
|---|---|---|
| Temperature/time regime | 115-117° | C./4.5 h; then |
| | 140-145° | C./4.5 h |
| Reaction Progress | | |

Monitored by detection of evolved ammonia.
Work-up
Ethylene glycol was removed from the reaction mixture by
distillation at about 18 mm Hg (pot Temperature 115-160° C.; vapor
temperature 94-98° C.). The distillation residue, which solidified upon
cooling to room temperature, was dispersed in 100 mL hot n-butanol,
cooled and filtered. The filter cake was then drained and dried to constant
weight. A second crop of crystals was harvested from the mother liquor
the following day. Altogether, 10.7 g. were obtained, melting at 133° C.
(literature 133-135° C.

| | |
|---|---|
| Yield | 50% |

### Utility Example 31

3-methyl-1-phenyl-5-pyrazolone

Starting Materials

| | | |
|---|---|---|
| Ethyl acetoacetate | 0.192 | mol (25 g) |
| Phenylhydrazine | 0.186 | mol (21 g) |
| Ethylene Glycol | 1.8 | mol (111 g) |

Operating Conditions

| Pressure | Atmospheric | |
|---|---|---|
| Temperature/time regime | 120° | C./2 h |
| Reaction Progress | | |
| Monitored by TLC | | |
| Work-up | | |

The reaction mass was allowed to cool to 40-45° C., quenched with
100 mL water, left undisturbed during 30 minutes, stirred for 3 hours and
filtered. The filter cake was washed with water, drained thoroughly and
dried at 70° C. to constant weight. 29.2 g. of ochre-colored crystals (m.p.
124.5-126.0° C.) were obtained. (Literature 127° C.)

| | |
|---|---|
| Yield | 91% |

### Utility Example 32

N-(2-hydroxyethyl)-2-oxazolidinone

Starting Materials

| | | |
|---|---|---|
| Diethly carbonate | 1.1 | mol (130 g) |
| Diethanolamine | 0.955 | mol (100 g) |
| Ethylene Glycol | 230 | g. |

Operating Conditions

| Pressure | Atmospheric | |
|---|---|---|
| Temperature/time regime | 98-103° | C./12.5 h |
| Reaction Progress | | |

Monitored by TLC and using a spot test for diethanolamine
(sodium nitroferricyanide/acetaldehyde/aqueous sodium carbonate)
Work-up
By-product ethanol, excess diethyl carbonate and ethylene glycol
(291 mL) were separated from product by distilling at atmospheric
pressure first and then at about 20 mm Hg at 25° C. The product fraction
weighed 123.9 g, (refraction index 1.482) (literature 1.483)

| | |
|---|---|
| Yield | 99% |

### Utility Example 33

N,N′-bis-[tris-(hydroxymethyl)methyl-] oxamide

Starting Materials

| | | |
|---|---|---|
| Diethly Oxalate | 0.125 | mol |
| tris(hydroxymethyl)aminomethane | 0.25 | mol |
| Ethylene Glycol | 100 | g. |

Operating Conditions

| Pressure | Atmospheric | |
|---|---|---|
| Temperature/time regime | 100-105° | C./6 h. |
| Reaction Progress | | |
| Monitored by TLC | | |
| Work-up | | |

The reaction mixture was cooled to 20° C., filtered under reduced
pressure, the filter cake washed with 50 mL absolute ethanol, drained well
and dried at 70-80° C. overnight. 34.0 g. of white crystals were obtained
with m.p. 216-217° C. (literature 216-218° C.)

| | |
|---|---|
| Yield | 92% |

### Utility Example 34

N-acetylglycine

Starting Materials

| | | |
|---|---|---|
| Ethyl Acetate | 0.5 | mol |
| Sodium glycinate | 0.5 | mol |
| Sodium methoxide (catalyst) | 0.25 | mol |
| Ethylene Glycol | 100 | g. |

Operating Conditions

| Pressure | Atmospheric | |
|---|---|---|
| Temperature/time regime | 75° | C./10 h. |
| Reaction Progress | | |
| Monitored by TLC | | |
| Work-up | | |

The reaction mixture was allowed to cool to room temperature, 300
mL water added, pH adjusted to 2-3 by adding 50.8 g. concentrated
aqueous hydrochloric acid, cooled to 10-15° C. and maintained at this
temperature during 1 h. Later it was filtered, the filter cake washed with
100 mL cold water, drained and dried overnight at 70-80° C. 30.3 g. of
yellowish white crystals were obtained with m.p. (dec.) 204-205° C.
(literature 206-208° C.)

| | |
|---|---|
| Yield | 52% |

### Utility Example 35

Hippuric acid

Starting Materials

| | | |
|---|---|---|
| Methyl benzoate | 0.5 | mol |
| Sodium glycinate | 0.5 | mol |
| Ethylene Glycol | 100 | g. |

Operating Conditions

| Pressure | Atmospheric | |
|---|---|---|
| Temperature/time regime | 114-127° | C./5 h. |
| Reaction Progress | | |

Monitored by TLC using Merck's silica gel plates and n-butanol:
ethanol:water at 2:2:1

Work-up

The reaction mixture was diluted with 450 mL water, extracted with
3 × 100 mL light petroleum ether, pH adjusted to 3.0 by addition of 53.5
g. concentrated aqueous hydrochloric acid, cooled to 10° C. Then it was
filtered under reduced pressure, the filter cake washed with 600 mL cold
water, drained and dried overnight at 70-80° C. 52.2 g. of white crystals
were obtained, m.p. 186-187° C. (literature 187-190° C.)

| | |
|---|---|
| Yield | 58% |

### Utility Example 36

N,N′-bis(4-methoxyphenyl) oxamide

Starting Materials

| Diethyl oxalate | 0.075 | mol |
|---|---|---|
| p-Anisidine | 0.151 | mol |
| Nitrogen | Only for system inertization | |
| Ethylene Glycol | 75 | g. |

Operating Conditions

| Pressure | Atmospheric | |
|---|---|---|
| Temperature/time regime | 120-125° | C./9 h. |
| Reaction Progress | | |

Monitored by TLC using Merck's silica gel plates, benzene-acetone
(3:1).
Work-up
The reaction mixture was cooled at 10° C. and filtered. The filter
cake was washed with cold methanol (120 mL), drained and dried at 90-
95° C. overnight. 16.9 g. of cream-colored crystals, m.p. 266-267° C.
were obtained (literature 270-271° C.)

| | |
|---|---|
| Yield | 75% |

### Utility Example 37

N-Benzylbenzamide

Starting Materials

| | | |
|---|---|---|
| Ethyl benzoate | 0.5 | mol (75 g) |
| benzylamine | 0.6 | mol (64 g) |
| Sodium methoxide (catalyst) | 15 | g. |
| Ethylene Glycol | 50 | g. |

Operating Conditions

| Pressure | Atmospheric | |
|---|---|---|
| Temperature/time regime | 110-120° | C./18 h. |
| Reaction Progress | | |

Monitored by TLC: Merck's silica gel plates; benzene-methanol
(20:3)
Work-up
The reaction mass was allowed to cool to 50° C., transferred to a
beaker, quenched with 400 mL. water and 150 mL methanol, stirred
during 15 minutes, its pH adjusted to 3 by adding 20 mL of concentrated
aqueous hydrochloric acid, cooled and filtered. The filter cake was then
drained, washed with 400 mL water, drained again and dried overnight at
70-75° C. 103 g. of white crystals (m.p. 102-103° C.) were obtained
(literature m.p. 105° C.)

| | |
|---|---|
| Yield | 97.5% |

1. An improved method applicable to the synthesis of compounds containing one or more OCN moieties from esters or ester-like compounds and ammonia (or its precursor) or an amine (or its precursor) or hydrazine (or its precursor) or a substituted hydrazine (or its precursor), or any amine-like compound in the presence of a diol or polyol.

2. An improved method applicable to the synthesis of amides or lactams through ammonolysis or aminolysis of esters, lactones, gem-diacyloxy derivatives, acetonides of alpha-hydroxyacids and other ester-like compounds in the presence of a diol or polyol.

3. An improved method for the preparation of compounds whose molecules contain 2 or more OCN moieties through reaction of esters or ester-like compounds with diamines or polyamines in the presence of a diol or polyol.

4. An improved method of claim 1 for the synthesis of compounds whose molecules contain 1 or more OCN moieties by ammonolysis or aminolysis of esters derived from dicarboxylic or polycarboxylic acids in the presence of a diol or polyol.

5. An improved method of claim 1 applicable to the synthesis of heterocyclic compounds whose molecules contain 1 or more OCN moieties—such as oxazolinones, oxazolidinones, oxazolidinediones, benzisoxazoles, benzimidazoles, pyrazolones, pyrazolidinediones, dihydrooxazinediones, barbituric acids, thiobarbituric acids, benzoxazoles, benzothiazoles, quinolones, pyridazinones, pyridones, hydroxypyrimidines, dihydroxypyrimidines, triazoles, etc—by reactions between an ester or diester and ammonia or an amine or diamine in the presence of a diol or polyol.

6-8. (canceled).

9. An improved method for obtaining compounds whose molecules contain 1 or more OCN moieties, as described in claim 1, which includes the use of a co-catalyst such as a metal alkoxide, a metal carbonate, a metal cyanide, an enzyme, a tertiary amine, a metal, or any transesterification catalyst.

10. An improved method for obtaining compounds whose molecules contain one or more OCN moieties, as described in claim 1, which employs pressures other than atmospheric.

11. An improved method for obtaining compounds whose molecules contain one or more OCN moieties, as described in claim 1, which involves the use of additives such as inert solvents and/or surface-active agents and/or antioxidants.

12. An improved method for obtaining compounds whose molecules contain one or more OCN moieties, as described in claim 1, which involves the use of more than one diol or polyol.

13. An improved method for obtaining compounds whose molecules contain one or more OCN moieties, as described in claim 1, wherein the alcoholic product is removed from the reaction mixture during the course of the reaction.

14. An improved method for obtaining compounds whose molecules contain one or more OCN moieties, as described in claim 1, which consists of two stages:
1) a transesterification reaction between an ester or ester-like compound and a diol or polyol (optionally catalyzed by sodium methoxide or other suitable catalysts) during or after which the alcohol may optionally be driven out, and
2) a reaction of the hydroxyester thereby obtained with the amine.

15. An improved method for obtaining compounds whose molecules contain one or more OCN moieties, as described in claim 1, which involves the use of more than one ester, or ester-like compound.

16. An improved method for obtaining compounds whose molecules contain one or more OCN moieties, as described in claim 1, which involves the use of more than one amine.

17. An improved method for obtaining compounds whose molecules contain one or more OCN moieties, as described in claim 1, which involves the use of more than one co-catalyst.

18. An improved method for obtaining compounds whose molecules contain one or more OCN moieties, as described in claim 1, which involves the use of more than one diol/polyol.

19. An improved method for obtaining compounds whose molecules contain one or more OCN moieties, as described in claim 1, which involves recycling of the mother liquor obtained after separation of the amide or amide-like product.
